(19) United States
(12) Patent Application Publication
(10) Pub. No.: US 2014/0212975 A1
(43) Pub. Date: Jul. 31, 2014

(54) COMPOSITIONS AND METHODS FOR BIOCULTURE OF WASABIA JAPONICA

(71) Applicant: Provitro Bioscience LLC, Mount Vernon, WA (US)

(72) Inventors: Jackie R Heinricher, Anacortes, WA (US); Randall Burr, Mount Vernon, WA (US); Jerrin Victor, Mount Vernon, WA (US)

(21) Appl. No.: 14/163,724

(22) Filed: Jan. 24, 2014

Related U.S. Application Data
(60) Provisional application No. 61/756,820, filed on Jan. 25, 2013.

(51) Int. Cl. A01H 4/00

(52) U.S. Cl. CPC A01H 4/008
USPC 435/430

(57) ABSTRACT

The present invention provides media, kits, systems, and methods for achieving large scale wasabi production within a short time via bioculture. The present invention for wasabi production results in shorter tuber development phase and higher yield.

### CROSS-REFERENCE TO RELATED APPLICATIONS

This application claims the benefit of U.S. provisional application No. 61/756,820 filed on Jan. 25, 2013, which is hereby incorporated by reference in its entirely for all purposes.

### TECHNICAL FIELD

This invention provides compositions, systems, and methods for efficient, rapid and large scale production of Wasabia japonica using bioculture.

### BACKGROUND

Wasabia japonica, a.k.a. wasabi or Eutrema japonica, is difficult to cultivate, which makes it quite expensive. Few places are suitable for large-scale natural wasabi cultivation, and cultivation is difficult even in ideal conditions. Due to its high cost, a common substitute is a mixture of horseradish, mustard, starch and green food coloring. Outside of Japan, it is rare to find real wasabi plants. Often packages are labeled as wasabi, but the ingredients do not actually include wasabi plant. In addition, many pathogens can infect Wasabia japonica, which make it harder to grow the plant. Previous wasabi tissue culture methods are very expensive and impractical to be used for large-scale production. Thus, there is a great need to identify compositions and methods for large-scale production of disease free Wasabia japonica.

### SUMMARY OF THE INVENTION

The present invention provides compositions, methods, kits, bioreactors, and systems for efficient and rapid propagation of Wasabia japonica at a large scale via bioculture.

In some embodiments, the present invention describes an automated, or semi-automated, low-cost system for the production of Wasabia japonica, which significantly increases the quantity and quality of Wasabia japonica plants, the number and size of the resulting plants, reduces the cost and shortens the cultivation time.

This invention provides novel compositions and an efficient and rapid system for mass propagation of Wasabia japonica in vitro.

In one embodiment, the present invention provides media for plant micropropagation. In some further embodiments, the media are used for micropropagation of Wasabia japonica.

In some embodiments, the media are initiation media, multiplication media, and rooting media, such as the Wasabi III media and CPR media, or combination thereof.

In some embodiments, the initiation media comprise Murashige & Skoog (MS) salts and sucrose. In some embodiments, the sucrose concentration is about 25 to 35 g/L, for example, about 30 g/L.

In some embodiments, the initiation media comprises at least one cytokinin and at least one auxin.

In some embodiments, the cytokinin is 6-Benzylaminopurine (BAP) or functional derivatives thereof, such as those described in Staden (A Comparison of the Effect of 6-Benzylaminopurine Derivatives on some Aspects of Plant Growth, Volume 29, Issue 1, pages 137-139, August 1973) and Dolezal et al. (Preparation and biological activity of 6-benzylaminopurine derivatives in plants and human cancer cells, Bioorg Med Chem. Feb. 1, 2006; 14(3):875-84. Epub Oct. 7, 2005), each is herein incorporated by reference in its entirety. In some embodiments, the BAP concentration in the initiation media is about 0.1 to 2 mg/L, for example, about 0.5 mg/L.

In some embodiments, the auxin is indole-3-acetic acid (IAA) or functional derivatives thereof, such as those described in Furkawa et al. (Effect of indole-3-acetic acid derivatives on neuroepithelium in rat embryos, J Toxicol Sci. August 2005; 30(3):165-74), Mujahid et al. (Production of indole-3-acetic acid and related indole derivatives from L-tryptophan by Rubrivivax benzoatilyticus JA2, Appl Microbiol Biotechnol. February 2011; 89(4):1001-8. doi: 10.1007/s00253-010-2951-2. Epub Oct. 24, 2010), and Chung et al. (Indole derivatives produced by the fungus Colletotrichum acutatum causing lime anthracnose and postbloom fruit drop of citrus FEMS Microbiology Letters 226 (2003) 23-30), each is herein incorporated by reference in its entirety. In some embodiments, the IAA concentration in the initiation media is about 0.1 to 5 mg/L, for example, about 0.5 mg/L.

In some embodiments, the initiation media do not comprise pyridoxine, nicotinic acid, riboflavin, or any derivatives thereof. In some embodiments, the initiation media do not comprise Na₂H₂PO₄.

In some embodiments, the initiation media are liquid, semi-liquid, solid, or semi-solid media. In some embodiments, the initiation media comprise about 4 to about 10 grams gelling agent, such as agar.

In some embodiments, the initiation media has a pH of about 5.0 to 6.0, for example, about 5.7.

In some embodiments, the multiplication media are similar to, or as the same as the initiation media.

In some embodiments, the rooting media comprise Murashige & Skoog (MS) salts and sucrose. In some embodiments, the sucrose concentration is about 25 to 35 g/L, for example, about 30 g/L. In some embodiments, the rooting media comprises at least one auxin. In some embodiments, the rooting media do not comprise any cytokinin.

In some embodiments, the auxin concentration in the rooting media is higher than the auxin concentration in the corresponding initiation media or the multiplication media used in tissue culture. In some embodiments, the auxin is indole-3-acetic acid (IAA) or functional derivatives thereof. In some embodiments, the IAA concentration in the rooting media is at least 2 mg/L, for example, about 2.5 mg/L.

In some embodiments, the rooting media comprise pyridoxine, nicotinic acid, riboflavin, and/or any derivatives thereof. In some embodiments, the concentration of pyridoxine in the rooting media is about 0.1 to 2 mg/L, such as about 0.5 mg/L. In some embodiments, the concentration of nicotinic acid in the rooting media is about 0.1 to 2 mg/L, such as about 0.5 mg/L. In some embodiments, the concentration of riboflavin in the rooting media is about 5 to 15 mg/L, such as about 5 mg/L. In some embodiments, the rooting media comprises Na₂H₂PO₄, for example, about 100-200 mg/L, such as abour 170 mg/L.

In some embodiments, the rooting media are liquid, semi-liquid, solid, or semi-solid media. In some embodiments, the rooting media comprise at least two types of gelling agents. In some embodiments, at least one gelling agent is agar or carrageenan. In some embodiments, the total concentration of gelling agents is about 5 to 10 g/L, for example, about 8 g/L. In some embodiments, the rooting media comprise about 4 g/L agar and about 4 g/L carrageenan.

In some embodiments, the rooting media has a pH of about 5.0 to 6.0, for example, about 5.7.

The present invention also provides kits for producing plants. In some embodiments, the kits are used for producing wasabi plants. In some embodiments, the kits comprise one or more initiation medium described herein, one or more multiplication medium as described herein, and/or one or more rooting medium as described herein.

The present invention further provides methods for producing plants. In some embodiments, the methods are used for producing wasabi plants.

In some embodiments, the methods of the present invention comprise (a) obtaining wasabi explant. In some embodiments, such methods comprise surface sterilizing the explant. In some embodiments, rhizome shoot tips can be used as wasabi explant for tissue culture. In some embodiments, the explant is taken from wasabi plants growing in a greenhouse.

In some embodiments, the methods further comprise (b) initiating shoot from the explant obtained in step (a) on an initiation medium. In some embodiments, the step is done in a container, e.g., a tube, wherein the container contains an initiation medium. In some embodiments, the initiation medium is a Wasabi III medium. The wasabi explant is cultured in the container until shoot tips start breaking and forming multiple shoots.

In some embodiments, the methods further comprise (c) multiplying the shoot initiated from step (b) on a multiplication medium. In some embodiments, the step comprises transferring the materials obtained from step (b) to a new container comprising a multiplication medium of the present invention. In some embodiments, the multiplication medium is a Wasabi III medium. In some embodiments, the multiplication medium is a solid medium. In some embodiments, the multiplication medium is a liquid medium. In some embodiments, cultures are maintained under standard growth condition, such as about 20 to 28° C. (for example, about 22-24° C.), under a day/nigh photoperiod (for example, a 16/8 day/night cycle). In some embodiments, the cultures are maintained on the multiplication medium for 1, 2, 3, 4, 5, 6, 7, 8, or more cycles. In some embodiments, each cycle lasts about 2-4 weeks, such as about 3 weeks. In some embodiments, the cultures are maintained on the multiplication medium for an additional 3 weeks.

In some embodiments, the methods further optionally comprise (c′) dividing the multiplied plant tissues into clumps. In some embodiments, each clump contains about 3 to 6 shoots. In some embodiments, each shoot is about 1-1.5 inch tall.

In some embodiments, the methods further comprise (d) transferring the multiplied shoots of step (c) or step (c′) on a rooting medium to produce a wasabi plant. In some embodiments, the rooting medium is a CPR medium. In some embodiments, the rooting medium is a solid medium. In some embodiments, the rooting medium is a liquid medium. In some embodiments, clumps are kept on the medium until individual plants with roots are developed, for example, after about 2-4 weeks.

In some embodiments of the present invention, in order to verify pathogen-free plants are produced, the methods further comprise testing for the presence or absence of one or more wasabi pathogen species after one or more cycles. In some embodiments, the pathogen species tested for is a bacteria species, fungal species and/or virus species.

In some embodiments, one or more steps described above are performed in a bioreactor, for example, a temporary immersion bioreactor. In some embodiments, the temporary immersion bioreactor is an ebb and flow bioreactor. In some embodiments, the multiplication step and/or rooting step is performed in the bioreactor. In some embodiments, when a bioreactor is used, one or more of the media mentioned above is in liquid or semi-liquid form. The size of the bioreactor can be any suitable size based on production requirements. For example, the bioreactor can be about 0.1 to about 20 L. The bioreactor can be placed under standard growth conditions, such as about 20 to 26° C. (for example, about 22-24° C.), and a day/nigh photoperiod (for example, a 16/8 day/night cycle). In some embodiments, every 1 g plant material is supported by about 100 ml medium. In some embodiments, the medium in the bioreactor is refreshed regularly or when needed. In some embodiments, the medium is refreshed about every one to about every four weeks (with each round called a “cycle”). In some embodiments, after each cycle the amount of biomass increases for about 1 to about 5 times. In some embodiments, the wasabi shoots multiplied in the bioreactor are transferred to rooting medium to perform step (d). In some embodiments, the rooting medium is a liquid medium.

In some embodiments of the present invention, the methods further comprise propagating the wasabi plants obtained to produce wasabi plants in vitro or in vivo.

The present invention also provides methods to produce important chemicals derived from wasabi plants, such as isothiocyanates. In some embodiments, the methods comprise producing wasabi plants by the tissue culture methods of the present invention, and then extracting the chemicals from the wasabi plants.

### DETAILED DESCRIPTION

### Definition

As used herein, the verb “comprise” as is used in this description and in the claims and its conjugations are used in its non-limiting sense to mean that items following the word are included, but items not specifically mentioned are not excluded.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

As used herein, the term “plant” refers to any living organism belonging to the kingdom Plantae (i.e., any genus/species in the Plant Kingdom). This includes familiar organisms such as but not limited to trees, herbs, bushes, grasses, vines, ferns, mosses and green algae. The term refers to both monocotyledonous plants, also called monocots, and dicotyledonous plants, also called dicots. For example, in some embodiments, the plant is a species in the Wasabia genus. In some embodiments, the plant is W. japonica.

As used herein, the term “plant part” refers to any part of a plant including but not limited to the shoot, root, stem, axillary buds, seeds, stipules, leaves, petals, flowers, ovules, bracts, branches, petioles, node, internodes, bark, pubescence, tillers, rhizomes, fronds, blades, pollen, stamen, microtubers, and the like.

As used herein, the term “germplasm” refers to the genetic material with its specific molecular and chemical makeup that comprises the physical foundation of the hereditary qualities of an organism.

As used herein, the phrase “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, the term “variety” or “cultivar” means a group of similar plants that by structural features and performance can be identified from other varieties within the same species. The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

As used herein, the term “genotype” refers to the genetic makeup of an individual cell, cell culture, tissue, organism (e.g., a plant), or group of organisms.

As used herein, the term “clone” refers to a cell, group of cells, a part, tissue, organism (e.g., a plant), or group of organisms that is descended or derived from and genetically identical or substantially identical to a single precursor. In some embodiments, the clone is produced in a process comprising at least one asexual step.

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “population” means a genetically homogeneous or heterogeneous collection of plants sharing a common genetic derivation.

As used herein, the term “bioreactor” refers to any vessel, device or system capable of holding, supporting and/or growing viable tissue. In other words, the term “bioreactor” as used herein may refer to a growth vessel that holds viable plant tissue, various other components internal or external to the growth vessel that are required for or aid the holding, supporting and/or growing of viable plant tissue, and any subsystem thereof.

As used herein, the phrase “temporary immersion bioreactor” refers to any bioreactor designed to temporarily wet a part or entire culture or plant tissue with nutrient medium (e.g., liquid or semi-liquid) followed by draining a part or all of the excess nutrient medium.

As used herein, a “plant propagation system” is a bioreactor for growing viable plant tissue.

### Wasabia

Wasabi, a.k.a. Wasabia japonica, Eutrema japonica, Japanese-horseradish, Japanischer Meerrettich, or gochunaengi, is a member of the Brassicaceae family. Although it is also called Japanese horseradish, it is not actually from the horseradish species of plants. Its root is used as a condiment and has an extremely strong flavor. Its hotness is more akin to that of a hot mustard than that of the capsaicin in a chili pepper, producing vapours that stimulate the nasal passages more than the tongue. The plant grows naturally along stream beds in mountain river valleys in Japan. The two main cultivars in the marketplace are W. japonica ‘Daruma’ and ‘Mazuma’, but there are many others, such as those described in Growing Edge (2005, The Best Of Growing Edge International 2000-2005, New Moon Publishing. p. 57. ISBN 978-0-944557-05-1), which is herein incorporated by reference in its entirety.

Wasabi is generally sold either as a root which is very finely grated before use, as dried powder in large quantities, or as a ready-to-use paste in tubes similar to travel toothpaste tubes. In restaurants the paste is prepared when the customer orders, and is made using a grater to grate the root; once the paste is prepared, it loses flavor in 15 minutes. Fresh wasabi leaves can be eaten, having the spicy flavor of wasabi roots. Because the burning sensations of wasabi are not oil-based, they are short-lived compared to the effects of chili peppers, and are washed away with more food or liquid. The sensation is felt primarily in the nasal passage and can be quite painful depending on amount taken.

Legumes (peanuts, soybeans, or peas) may be roasted or fried, then coated with wasabi powder mixed with sugar, salt, or oil and eaten as a crunchy snack. Inhaling or sniffing wasabi vapor has an effect like smelling salts, a property exploited by researchers attempting to create a smoke alarm for the deaf. One deaf subject participating in a test of the prototype awoke within 10 seconds of wasabi vapor being sprayed into his sleeping chamber.

Wasabi is difficult to cultivate, and that makes it quite expensive. Due to its high cost, a common substitute is a mixture of horseradish, mustard, starch and green food coloring. Outside of Japan, it is rare to find real wasabi plants. Often packages are labeled as wasabi, but the ingredients do not actually include wasabi plant. Although the taste is similar between wasabi and horseradish, they are easily distinguished. In Japan, horseradish is referred to as “western wasabi”. In the United States, true wasabi is generally found only at specialty grocers and high-end restaurants.

The chemical in wasabi that provides for its initial pungency is the volatile allyl isothiocyanate, which is produced by hydrolysis of natural rhizome thioglucosides (conjugates of the sugar glucose, and sulfur-containing organic compounds); the hydrolysis reaction is catalyzed by myrosinase and occurs when the enzyme is released on cell rupture caused by maceration—e.g., grating—of the plant's rhizome.

The unique flavor of wasabi is a result of complex chemical mixtures from the broken cells of the rhizome, including those resulting from the hydrolysis of thioglucosides into glucose and methylthioalkyl isothiocyanates: 6-methylthiohexyl isothiocyanate, 7-methylthioheptyl isothiocyanate, and 8-methylthiooctyl isothiocyanate. Research has shown that such isothiocyanates inhibit microbe growth, perhaps with implications for preserving food against spoilage and suppressing oral bacterial growth.

Few places are suitable for large-scale wasabi cultivation, and cultivation is difficult even in ideal conditions. In Japan, wasabi is cultivated mainly in these regions: Izu peninsula, located in Shizuoka prefecture, Nagano prefecture, and Iwate prefecture.

There are also numerous artificially cultivated facilities as far north as Hokkaido and as far south as Kyushu. As the demand for real wasabi is very high, Japan has to import a large amount of it from China, Ali Mountain of Taiwan, and New Zealand. In North America, a handful of companies and small farmers are pursuing the trend by cultivating Wasabia japonica.

Wasabi is often grated with a metal oroshigane, but, some prefer to use a more traditional tool made of dried sharkskin with fine skin on one side and coarse skin on the other. A hand-made grater with irregular teeth can also be used. If a shark-skin grater is unavailable, ceramic is usually preferred.

Numerous studies have demonstrated that Wasabia japonica contains natural chemicals which are highly efficacious against a variety of cancers, such as breast, prostate, colon, lung, leukemia, pancreas esophagus, bladder and others. These chemicals are known as isothiocyanates which arise from the enzymatic breakdown of glucosinolate molecules found in intact cells. When Wasabi cells are disrupted (i.e. macerated) the glucosinolates come contact the myrosinase enzyme which catalyses the conversion to isothiocyanates.

Chadwick et al. (1993) explained that plants can be established from either offshoot cuttings taken from a previous crop, seedlings grown from seed, or tissue-cultured plantlets. Japanese farmers normally propagate wasabi using offshoots taken from the main stems of mature plants. Hartmann et al. (1990) reported large genetic advances can be made in a single step by selecting a single unique superior plant from a seedling population and reproducing it asexually by vegetative propagation. The resulting population of plants has the same basic genotype as the original seedling plant. These plants are also highly uniform phenotypically.

### Media

The present invention provides media comprising compounds with unique types, concentrations, and combinations. In some embodiments, the medium is a liquid, semi-liquid, solid or semi-solid medium.

In some embodiments, liquid cultures offer several advantages. The liquid cultivation saves time, because it enables replacement of the full medium in the vessel containing multiple explants be made at once, instead of individual transfers of single plant. In addition, a liquid culture results in increased shoot length because a larger area of the explant can get in contact with the medium.

The physical state of the media can vary by the incorporation of one or more gelling agents. Any gelling agent known in the art that is suitable for use in plant tissue culture media can be used. Agar is most commonly used for this purpose. Examples of such agars include Agar Type A, E or M and Bacto™ Agar. Other exemplary gelling agents include carrageenan, gellan gum (commercially available as PhytaGel™, Gelrite® and Gelzan™), alginic acid and its salts, and agarose. Blends of these agents, such as two or more of agar, carrageenan, gellan gum, agarose and alginic acid or a salt thereof also can be used. In some embodiments, no gelling agent or very little gelling agent is used for a liquid medium.

In some embodiments, the media comprise one or more minimum nutrition necessary for plant growth, such as amino acids, macroelements, microelements, aluminum, boron, chlorine (chloride), chromium, cobalt, copper, iodine, iron, lead, magnesium, manganese, molybdenum, nitrogen (nitrates), potassium, phosphorous (phosphates), silicon, sodium, sulphur (sulphates), titanium, vanadium, zinc, inositol and undefined media components such as casein hydrolysates or yeast extracts. For example, the media can include any combination of NH₄NO₃; KNO₃; Ca(NO₃)₂; K₂SO₄; MgSO₄; MnSO₄; ZnSO₄; K₂SO₅; CuSO₄; CaCl₂; KI; CoCl₂; H₃BO₃; Na₂MoO₄; KH₂PO₄; FeSO₄; Na₂EDTA; Na₂H₂PO4; inositol (e.g., myo-inositol); thiamine; pyridoxine; nicotinic acid; glycine; and riboflavin. It is known to those in the art that one or more components mentioned above can be omitted without affecting the function of the media.

The media can comprise one or more carbon source, such as a sugar. Non-limiting exemplary sugars include sucrose, glucose, maltose, galactose and sorbitol or combinations thereof.

In some embodiments, the media can comprise one inorganic salts, growth regulators, carbon source, and/or vitamins. In some embodiments, at least one of the vitamins is provided by the Murashige and Skoog medium salts (Murashige and Skoog, 1962), and/or functional variations thereof.

The media further comprise one or more effective amount of plant growth regulators. Examples of plant growth regulators include plant hormones, such as auxins and compounds with auxin-like activity, cytokinins and compounds with cytokinin-like activity. The term “cytokinin” refers to a class of plant growth regulators that are characterized by their ability to stimulate cell division and shoot organogenesis in tissue culture. Non-limiting examples of cytokinins include, N⁶-benzylaminopurine (BAP) (a.k.a. N⁶-benzyladenine (BA)), meta-topolin, zeatin, kinetin, thiadiazuron (TDZ), meta-topolin, 2-isopentenyladenine (a.k.a., 6-γ-γ-(dimethylallylamino)-purine or 2ip), adenine hemisulfate, dimethylallyladenine, 4-CPPU (N-(2-chloro-4-pyridyl)-N′-phenylurea)), and analogs thereof. The term “auxin” refers to a class of plant growth regulators that are characterized principally by their capacity to stimulate cell division in excised plant tissues. In addition to their role in cell division and cell elongation, auxins influence other developmental processes, including root initiation. Non-limiting examples of β-naphthoxyacetic acid (NAA), 2,4-Dichlorophenoxy acetic acid (2,4-D), indole-3-butyric acid (IBA), indole-3-acetic acid (IAA), picloram, and analogs thereof. More cytokinins and auxins are described in WO2011100762, U.S. Pat. No. 5,211,738, US20100240537, US20060084577, US20030158043, and Aremu et al., 2011, which are incorporated by reference in their entireties. In some embodiments, the cytokinin is BAP or any functional variant thereof. In some embodiments, the auxin is IAA or any functional variant thereof.

In some embodiments, other plant growth regulators can be added in the media to improve cell growth and development.

Exemplary concentrations of the components described above are shown in Table 1. The concentrations of these components can be adjusted based on plant species, tissue type, and purposes, etc, without substantially affecting the media function.

TABLE 1
Exemplary Concentrations

| | Concentrations |
|---|---|
| | (mg/L in all unless otherwise |
| Component | noted) |
| NH₄NO₃ | about 800-about 2500 |
| KNO₃ | about 900-about 3000 |
| Ca(NO₃)₂ | 0-about 800 |
| K₂SO₄ | 0-about 800 |
| MgSO₄ | about 150-about 550 |
| MnSO₄ | about 8.0-about 26.0 |
| ZnSO₄ | about 4.0-about 12.0 |
| CuSO₄ | about 0.010-about 0.4 |
| CaCl₂ | about 200-about 660 |
| KI | about 0.4-about 1.5 |
| CoCl₂ | about 0.010-about 0.4 |
| H₃BO₃ | about 3.0-about 9.0 |
| Na₂MoO₄ | about 0.10-about 0.4 |
| KH₂PO₄ | about 80-about 250 |
| FeSO₄ | about 25-about 90 |
| Na₂EDTA | about 35-about 120 |
| Na₂H₂PO₄ | About 0-250 |
| myo-Inositol | about 50-about 150 |
| Thiamine | about 0.2-about 0.6 |
| Sugar | about 10 g/L-about 100 g/L |

As used herein and in the claims, where the term “about” is used with a numerical value, the numerical value may vary from the explicit number. For example, the variation will be ±30%, ±20%, ±10%, ±5%, ±4%, ±3% ±2%, ±1% or less.

Optionally, the media further comprise one or more buffering agent. The buffering agent can buffer the salt concentration and/or the pH in the medium. For example, the buffering agent can maintain the pH of the liquid mixture so the pH is kept around about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5 about 6.6, about 6.7, about 6.8, about 6.9, or about 7.0. For example, the pH of the liquid mixture is in a range between about 5.0 to about 7.0. In some embodiments, the buffering agent is 2-(N-morpholino)ethanesulfonic acid (MES), Adenosine deaminase (ADA), piperazine-N,N′-bis(2-ethanesulfonic acid) (PIPES), N-(2-Acetamido)-2-aminoethanesulfonic acid (ACES), Cholamine chloride, etc. In some embodiments, the pH of the medium is maintained at about 5.0 to 6.0, for example, about 5.7.

If present in a media, each cytokinin can be present in an amount from about 0.001 mg/L-about 10 mg/L and all amounts in between. For example, the concentration of a cytokinin is about 0.001, 0.01, 0.1, 1, 2, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 9, or about 10 mg/L. In some embodiment, at least one cytokinin is BAP, and its concentration is about 0.1 to 5 mg/L, for example, about 0.5 mg/L.

If present in a media, each auxin can be present in an amount from about 0.01 mg/L-about 10 mg/L and all amounts in between. For example, the concentration of an auxin is about 0.001, 0.01, 0.02, 0.03, 0.04, 0.05, 0.06, 0.07, 0.08, 0.09, 0.1, 0.5, 1, 2, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 9, or about 10 mg/L. In some embodiments, at least one auxin is IAA. In some embodiments, IAA is presented in an initiation medium or a multiplication medium, and the concentration is about 0.1 to 2 mg/L, for example, about 0.5 mg/L. In some embodiments, IAA is presented in a rooting medium, and the concentration is higher than that in the corresponding initiation medium or multiplication medium. In some embodiments, the IAA concentration in a rooting medium is about 1 to 5 mg/L, for example, about 2.5 mg/L.

In some embodiments, the present invention provides several types of media that are used in wasabi in vitro micropropagation.

The first type of media, referred herein as the initiation media, is similar to or essentially the same as the Murashige and Skoog medium (Murashige and Skoog, 1962), but comprise at least one auxin and at least one cytokinin. In some embodiments, the sucrose concentration in the propagation and multiplication media is about 20-40 g/L. In some embodiments, at least one auxin is IAA. In some embodiments, at least one cytokinin is BAP. In some embodiments, the concentration of BAP is about 0.1 to about 5 mg/L, for example, about 0.5 mg/L. In some embodiments, the concentration of IAA is about 0.1 to about 2 mg/L, for example, about 0.5 mg/L. In some embodiments, the media comprise at least one gelling agent such as agar. In some embodiments, the concentration of the gelling agent is about 4 to about 10 grams, for example, about 7 grams. In some embodiments, the initiation media do not contain Na₂H₂PO₄. In some embodiments, the initiation media do not contain pyridoxine, nicotinic acid, and/or riboflavin.

The second type of media, referred herein as the micropropagation media or multiplication media, are as the same as, or similar to the initiation media. In some embodiments, the sucrose concentration in the propagation and multiplication media is about 20-40 g/L. In some embodiments, at least one auxin is IAA. In some embodiments, at least one cytokinin is BAP. In some embodiments, the concentration of BAP is about 0.1 to about 5 mg/L, for example, about 0.5 mg/L. In some embodiments, the concentration of IAA is about 0.1 to about 2 mg/L, for example, about 0.5 mg/L. In some embodiments, the media comprise at least one gelling agent such as agar. In some embodiments, the media comprise at least one gelling agent such as agar. In some embodiments, the concentration of the gelling agent is about 4 to about 10 grams, for example, about 7 grams. In some embodiments, the multiplication media do not contain Na₂H₂PO₄. In some embodiments, the multiplication media do not contain pyridoxine, nicotinic acid, and/or riboflavin.

The third type of media, referred herein as the rooting media, are similar to or essentially the same as the Murashige and Skoog medium, but comprise at least one auxin and do not comprise any cytokinin. In some embodiments, the sucrose concentration in the propagation and multiplication media is about 20-40 g/L. In some embodiments, the concentration of IAA is higher than the corresponding multiplication media used in the multiplication step. In some embodiments, the concentration of IAA is about 1 to about 5 mg/L, for example, about 2.5 mg/L. In some embodiments, the concentration of the gelling agent is about 4 to about 10 grams, for example, about 8 grams. In some embodiments, the at least two gelling agents are included. In some embodiments, at least one gelling agent is agar, and the other gelling agent is carrageenan. In some embodiments, the rooting media contain Na₂H₂PO₄. In some embodiments, the initiation media contain pyridoxine, nicotinic acid, and/or riboflavin.

### Plant Propagation Bioreactor

Bioreactors can be used for plant micropropagation to more efficiently increase shoot mass than in stationary cultures. Bioreactors offer a promising way of scaling-up micropropagation processes, making it possible to work in large containers with a high degree of control over culture parameters (e.g., pH, aeration, oxygen, carbon dioxide, hormones, nutrients, etc.). Bioreactors are also compatible with the automation of micropropagation procedures, utilizing artificial intelligence, which reduces production costs.

In some embodiments, a temporary immersion bioreactor (TIB, a.k.a. temporary immersion system (TIS) or “Ebb and Flow” bioreactor) is used. Non-limiting examples of temporary immersion bioreactors include nutrient mist bioreactors, tilting and rocking vessels, twin flask system, or single containers with at least two compartments, such as Recipient for Automated Temporary Immersion (RITA®).

In some embodiments, the temporary immersion bioreactor is disposable. In some embodiments, the temporary immersion bioreactor is reusable.

In some embodiments, the temporary immersion bioreactor involves a wetting and drying cycle which occurs periodically in a predetermined period of time and hence it can also be termed as periodic, temporary immersion. In some embodiments, the temporary immersion bioreactor has a mere up-and-down motion of the nutrient medium without renewal. In some embodiments, renewal of nutrient medium in the bioreactor is involved.

Temporary immersion bioreactors provide an excellent way of using liquid medium at the same time controlling the gaseous environment. Moreover, it can provide the possible automation of the production system which facilitates low production costs. In other words, increasing the rate of growth and multiplication by using bioreactors more plants per unit area of the growth room are produced, which reduces the cost per plant per unit space of growth room. Liquid culture bioreactors are mainly suitable for the large-scale production of small size somatic embryos, growth of bulb, corms, micro tubers, compact shoot cultures etc. Major features of a temporary immersion bioreactor are:
Reduction of hyperhydricity, compared with that of permanent immersion, is the major achievement of a temporary immersion system. As plants are immersed in the medium for short time, the physiological disorders are reduced and the plants become healthier.
Plant growth and development can be controlled by manipulating the frequency and duration of immersion in liquid medium.
Plant growth is improved because during every immersion the plant is in direct contact with the medium and a thin film of liquid covers the plant throughout the interval period.
Air vents attached to the vessel prevent the cultures from contamination.
Due to the lack of agitation or aeration, the mechanical stress on plant tissues are generally low compared with the other bioreactor systems.

Temporary immersion bioreactors, which represent simple plastic vessels with medium (e.g., liquid, semi-liquid, etc) moving from one side to another every several minutes, can be used to generate microtubers. This temporary immersion system has been shown to stimulate shoot multiplication in many plant species. For example, the multiplication rates for sugarcane and pineapple were 6 and 3-4 times, respectively, higher compared with the rates obtained in liquid or solid media (Lorenzo et al., 1998; Escalona et al., 1999). In some embodiments, the bioreactor used in the present invention is a bioreactor described in International Patent Application No. PCT/US2012/047622, which is incorporated by reference in its entirety.

Other non-limiting examples of plant micropropagation systems include those described in U.S. Pat. Nos. 3,578,431; 4,320,594; 4,669,217; 4,908,315; 4,934,096; 5,049,505; 5,088,231; 5,104,527; 5,119,588; 5,139,956; 5,171,683; 5,184,420; 5,186,895; 5,212,906; 5,225,342; 5,558,984; 5,597,731; and U.S. Pat. No. 6,753,178. More examples of plant micropropagation systems can be found in Etienne et al. (Bioreactors in coffee micropropagation, Braz. J. Plant Physiol., 18(1):45-54, 2006); Ziv (Bioreactor Technology for Plant Micropropagation Horticultural Reviews, Volume 24, Edited by Jules Janick ISBN 0-471-33374-3); and Paek et al. (Application of bioreactors for large-scale micropropagation systems of plants, In Vitro Cell. Dev. Biol.-Plant 37:149-157, March-April 2001). It is understood that plant propagation systems that can be used in the present invention includes those derived from the ones described above by adding or reducing one or more parts/features of the systems known to one skilled in the art.

### Methods for Wasabi Bioculture

The present application provides methods for wasabi tissue culture. The methods enable a massive production of wasabi plants within a short time, at a low cost. The methods can be conducted with or without a bioreactor.

In some embodiments, the methods comprise (a) obtaining wasabi explant. Although other plant parts may also work, Rhizome shoot tips are ideal starting material as explants. The explants can be treated to substantially reduce the chance of contamination. Any suitable methods can be used, such as the methods described in Quazi et al. 1978; Al-Taleb 2011; and Plant Tissue Culture—Theory and Practice, a Revised Edition, Chapter 15, 1996; 2006 OEPP/EPPO, Bulletin OEPP/EPPO Bulletin 36, 187. In some embodiments, commercial bleach can be used. For example, explant can be sterilized in about 5%, 10%, 15% or more commercial bleach for about 10 minutes, 20 minutes, 30 minutes, or more depending on the condition of the explant. In some embodiments, to further reduce the chance of contamination, explant can be cut into small piece, such as about 3 mm, about 5 mm, or more in length. The small pieces can be rinsed again once, twice, or more in 1% commercial bleach solution and then placed on an initiation medium. The initiation medium can be any suitable medium as described herein.

This step is completed when shoot tips start breaking and forming multiple shoots from the explant, which usually takes about 2 to 3 months after the initiation step starts. During the process, explant can be sub cultured on a fresh initiation medium every 3 to 4 weeks.

The multiple shoots initiated from the explant can be dived into small clumps, for example, clumps of 2 to 3 shoots each and transferred to a multiplication medium as described herein. This step can be conducted in a bioreactor.

Any suitable growth condition can be used. In some embodiments, the plants are grown under about 20-28° C. (e.g., about 22-24° C.), day-night cycle (e.g., 16/8 hour day/night), with a photon flux density of about 10-150 μmol/m²/s.

In some embodiments, a temporary immersion bioreactor is used. In some embodiments, in a single cultivation cycle, it takes about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more to fill the cultivation chamber of the bioreactor in which the plants are grown with a predetermined amount of liquid or semi-liquid medium. The medium is kept in the chamber for about 1, 2, 3, 4, 5, 6, 7, 8, 9, 10, 15, 20, 25, 30, 35, 40, 45, 50, 55, 60 minutes or more, and then drained from the chamber. In some embodiments, it takes about 0.1, 0.2, 0.3, 0.4, 0.5, 1, 1.5; 2, 2.5, 3, 3.5, 4, 4.5, 5, 5.5, 6, 7, 8, 9, 10, 15, 20, 25, 30 minutes or more minutes to drain the medium out of the chamber. Then optionally the chamber is dried for about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more minutes. In some embodiments, the cultivation cycle described above takes about 1, 2, 3, 4, 5, 6, 7, 8, 9, 10, 15, 20, 25, 30, 35, 40, 45, 50, 55, 60 minutes or more.

In some embodiments, a relatively small amount of liquid medium is used, such as about 25-100 ml of liquid medium per bioreactor. Advantages of using a relatively small amount of liquid medium include, but are not limited to, better control of temporal immersion of the explants, preventing drowning of the explains, and reducing chances of contamination.

In some embodiments, the liquid medium in the bioreactors is changed with fresh one every 1 week, 2 weeks, 3 weeks, 4 weeks or more, each of which is called a growth cycle (or cycle). Advantages of using refreshed liquid medium include, but are not limited to, providing more nutrients to plants, removing accumulated detrimental chemicals in the medium due to plant metabolism, and reducing chances of contamination.

In some embodiments, an oscillating rack system is used to move liquid from one side to another. In some embodiments, the oscillation cycle is about once per two minutes, once per minute, one and a half per minute, twice per minute or more. In some embodiments, the oscillating rack system is used in the initiation and/or multiplication step. A non-limiting example of oscillating rack system is described in International Patent Application No. PCT/US2012/047622, which is incorporated herein in its entirety including any figures therein.

In some embodiments, the wasabi plant tissue biomass are multiplied for about 1.5 times, about 2 times, about 2.5 times, about 3 times, about 3.5 times, about 4 times, about 4.5 times, about 5 times, about 5.5 times, about 6.0 times, about 6.5 times, about 7 times, about 7.5 times, about 8 times, or more during each growth cycle.

Any suitable plant, plant part, plant tissue culture, or plant cell can be used as the explant. In some embodiments, the explant is pathogen-free, e.g., bacteria-free, fugi-free and/or virus-free. In some embodiments, the explant is a wasabi rhizome shoot tip. In some embodiments, the explant is taken from a plant of about 1 week old, about 2 weeks old, about 3 weeks old, about 1 month old, about 2 month old, about 3 months old, about half year old, about 1 year old, or more.

The explant can be any segment or collection of cells from apical meristems, terminal buds, axillary buds, adventitious buds, accessory buds, pseudo-terminal buds, cambium, lateral meristem, lateral bud, vegetative buds, reproductive buds, mixed buds, shoot segments, shoot apices, stem segments, immature nodal sections from stems, lateral shoots, seedlings, seeds, shoots starting to rise from the ground, immature flower buds, inflorescences, crown segments, leaf segments, roots, rhizome, or any part thereof.

The multiplied shoots are then transferred to a rooting medium as described herein. Optionally, the multiplied shoots are divided into clumps of about 3 to 6 shoots before the transfer. In some embodiments, the shoots are about 1 to 1.5 inch high. It usually takes about 3-4 weeks for the shoots to develop roots. Once the roots are formed, the plants can be transfer to either in vitro or in vivo conditions for further growth.

The methods described herein can be further modified and optimized, depending on the purposes, goals, and other factors that may affect wasabi tissue culture. For example, factors affecting wasabi tissue culture are disclosed in Rodriguez (2000, Thesis, Micropragation of Wasabi and Identification of Pathogens Affecting Plant Growth and Quality, Simon Fraser University), Sparrow (2001, Evaluation and development of Wasabi Production for the East Asian Market), Craigie (2002, Thesis, Yield and quality response of wasabi (Wasabia japonica (Miq.) Matsumara) to nitrogen and sulphur fertilizers, Lincoln University of New Zealand), Chadwick et al. (1993. The botany, uses and production of Wasabia japonica (Miq.) (Cruciferae) Matsum. Economic Botany 47(2),113-135), each of which is incorporated herein by reference in its entirety for all purposes.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1

### In Vitro Initiation

Rhizome shoot tips were used as explants. The explants were taken from greenhouse grown plants. The size of the explants can vary from about 10 to about 20 mm in length. The explants were washed in soap water and surface sterilized in about 10% solution of commercial bleach for about 30 minutes. After that the shoot tips were further cut to small pieces of about 5 mm each and rinsed twice in about 1% commercial bleach solution. For culture initiation individual explants were then moved to test tubes containing Wasabi III solid medium (see Table 2 below).

The cultures were kept under standard condition (22-24° C., 16/8 hours day/night photoperiod) and every 3 to 4 weeks were sub-cultured into fresh Wasabi III medium. When the shoot tips started breaking and forming multiple shoots the initiation step was completed. This was normally taking place in about 2 to about 3 months after starting the cultures.

TABLE 2
Wasabi III medium

| Chemical | mg/L |
|---|---|
| NH4NO3 | 1,650.00 |
| KNO3 | 1,900.00 |
| MgSO4 | 370.00 |
| MnSO4 | 16.90 |
| ZnSO4 | 8.60 |
| CuSO4 | 0.03 |
| CaCl2 | 440.00 |
| KI | 0.83 |
| CoCl2 | 0.03 |
| H3BO3 | 6.20 |
| Na2MoO4 | 0.25 |
| KH2PO4 | 170.00 |
| FeSO4 | 27.80 |
| Na2EDTA | 37.30 |
| Na2H2PO4 | — |
| myo-Inositol | 100.00 |
| Thiamine | 0.40 |
| Pyridoxine | — |
| Nicotinic acid | — |
| Riboflavin | — |
| IAA | 0.50 |
| BAP | 0.50 |
| Sugar | 30,000.00 |
| Agar | 7,000.00 |
| PH | 5.70 |

### Example 2

### In Vitro Multiplication on Solid Medium

For multiplication the multiple shoot cultures obtained in Example 1 were firstly divided into small clumps of 2 to 3 shoots each and transferred to boxes or plastic containers containing the same Wasabi III solid medium. Cultures were maintained under standard condition (22-24° C. 16/8 hours photoperiod) for an additional 3 weeks. Every 3 to 4 weeks the cultures were divided into smaller clumps of 2 to 3 shoots and transferred to fresh medium.

### Example 3

### In Vitro Rooting on Solid Medium

Cultures were further divided into clumps of 3 to 6 shoots approximately 1.5 inch tall and transferred onto “CPR” medium (see Table 3 below). Cultures were kept for additional 4 weeks until individual plants with roots were fully developed.

TABLE 3
CPR medium

| Chemical | mg/L |
|---|---|
| NH4NO3 | 1,650.00 |
| KNO3 | 1,900.00 |
| MgSO4 | 370.00 |
| MnSO4 | 16.90 |
| ZnSO4 | 8.60 |
| CuSO4 | 0.03 |
| CaCl2 | 440.00 |
| KI | 0.83 |
| CoCl2 | 0.03 |
| H3BO3 | 6.20 |
| Na2MoO4 | 0.25 |
| KH2PO4 | 170.00 |
| FeSO4 | 27.80 |
| Na2EDTA | 37.30 |
| Na2H2PO4 | 170.00 |
| myo-Inositol | 100.00 |
| Thiamine | 0.4 |
| Pyridoxine | 0.5 |
| Nicotinic acid | 0.5 |
| Riboflavin | 10.0 |
| IAA | 2.5 |
| BAP | — |
| Sugar | 30,000.00 |
| Agar | 4,000.00 |
| Carrageenan | 4,000.00 |
| PH | 5.70 |

### Example 4

### In Vitro Multiplication in Liquid Medium

Wasabi cultures were also multiplied in liquid medium using temporary immersion bioreactor vessels. The size of bioreactor varies from 0.1 to 20 L depending on production requirements. Bioreactors were inoculated with Wasabi material produced in tubes or boxes. Usually a gram of material was introduced for each 100 ml of Wasabi III liquid medium. Bioreactors were kept under standard conditions (22-24° C. and 16/8 hours day/night photoperiod). Media were refreshed every one to four weeks. After each cycle the amount of biomass increased between 2 to 5 times. After several multiplication cycles the shoots were be further subjected to in vitro rooting under solid or liquid conditions.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A set of media for producing wasabi plants or plant parts wherein the set of media comprises:
(1) one or more initiation medium;
(2) one or more multiplication medium; and
(3) one or more rooting medium,
wherein the initiation medium and/or the multiplication medium comprises 6-Benzylaminopurine (BAP) or any functional derivative, and indole-3-acetic acid (IAA) or any functional derivative thereof, and the rooting medium comprises IAA and does not comprise any cytokinin.

2. The set of media of claim 1, wherein the initiation medium and the multiplication medium are the same.

3-5. (canceled)

6. The set of media of claim 1, wherein the BAP in the initiation medium and/or the multiplication medium has a concentration of about 0.5 mg/L.

7. The set of media of claim 1, wherein the IAA in the rooting medium has a concentration higher than the IAA concentration in the initiation medium and/or the multiplication medium.

8. The set of media of claim 7, wherein the IAA concentration in the rooting medium is about 2 mg/L to about 5 mg/L, and the IAA concentration in the initiation medium and/or the multiplication medium is about 0.1 mg/L to less than 2 mg/L.

9. The set of media of claim 8, wherein the IAA concentration in the rooting medium is about 2.5 mg/L, and the IAA concentration in the initiation medium and/or the multiplication medium is about 0.5 mg/L.

10. The set of media of claim 1, wherein the rooting medium comprises Pyridoxine, Nicotinic acid, and/or Riboflavina, while the initiation medium and/or the multiplication medium does not comprise Pyridoxine, Nicotinic acid or Riboflavina.

11-12. (canceled)

13. A method for producing wasabi plant in vitro comprising:
(a) obtaining a wasabi explant;
(b) initiating shoot from the explant obtained in step (a) on an initiation medium;
(c) multiplying the shoot initiated from step (b) on a multiplication medium; and
(d) transferring the multiplied shoots of step (c) on a rooting medium to produce a wasabi plant with root;
wherein the wasabi explant is obtained from a rhizome shoot tip, and wherein the initiation medium and/or the multiplication medium comprises 6-Benzylaminopurine (BAP) or any functional derivative, and indole-3-acetic acid (IAA) or any functional derivative thereof, and the rooting medium comprises IAA and does not comprise any cytokinin.

14-15. (canceled)

16. The method of claim 13, wherein the wasabi explant is about 10 to 20 mm in length, and wherein the explant is washed and surface sterilized prior to the initiating step.

17-18. (canceled)

19. The method of claim 16, wherein the explant is further cut to smaller pieces of about 5 mm each and rinsed at least once in 1% bleach.

20. The method of claim 13, wherein all media are solid media.

21. The method of claim 13, wherein said multiplying medium is liquid medium.

22. The method of claim 13, wherein the initiation step (b) comprises sub-culturing the explant in a fresh medium every 3 to 4 weeks until shoot tip starts breaking and forming multiple shoots.

23. (canceled)

24. The method of claim 13, wherein multiple shoots form after step (b), and the multiple shoots are divided into small clumps of about 2 or more shoots each and transferred to a multiplication medium to perform the step (c).

25-30. (canceled)

31. The method of claim 13, wherein at least one step of the method is performed in a bioreactor.

32. The method of claim 31, wherein the bioreactor is a temporary immersion bioreactor.

33. The method of claim 32, wherein the temporary immersion bioreactor is an ebb and flow bioreactor.

34-35. (canceled)

36. The method of claim 3, wherein every 1 gram of wasabi tissue is supported by about 100 ml medium.

37-38. (canceled)

39. The method of claim 31, wherein the medium is refreshed every one to four weeks to constitute a growth cycle.

40. The method of claim 39, wherein the bioreactor enables wasabi biomass to increase about 2 to 5 times in each growth cycle.

41-43. (canceled)
